(19) United States
(12) Patent Application Publication
(10) Pub. No.: US 2003/0223938 A1
(43) Pub. Date: Dec. 4, 2003

(54) Polyvalent nanoparticles

(76) Inventors: John O. Nagy, Bozeman, MT (US); Robert F. Bargatze, Bozeman, MT (US); John W. Jutila, Bozeman, MT (US); Jim E. Cutler, New Orleans, LA (US); Yongmoon Han, Bozeman, MT (US); Pati M. Glee, Bozeman, MT (US); David Pascual, Bozeman, MT (US)

Correspondence Address: MORGAN LEWIS & BOCKIUS LLP, 1111 PENNSYLVANIA AVENUE NW, WASHINGTON, DC, 20004, US

(21) Appl. No.: 10/412,685

(22) Filed: Apr. 14, 2003

Related U.S. Application Data
(63) Continuation of application No. PCT/US01/42712, filed on Oct. 15, 2001.
(60) Provisional application No. 60/239,874, filed on Oct. 13, 2000.

(51) Int. Cl. A61L 9/04; A61K 9/14; A61K 31/715; A61K 39/12; A61K 39/02

(52) U.S. Cl. 424/46; 424/489; 424/204.1; 424/235.1; 514/54

(57) ABSTRACT

The present invention relates to nanoparticles comprised of a carrier, particularly polymerized lipids, and ligands displayed on the carrier, wherein the ligands form a polyvalent binding unit that is effective to produce a specific interaction between the nanoparticle and receptors on a target, particularly under physiologically relevant shear conditions.

### RELATED APPLICATIONS

This application claims priority to U.S. application Ser. No. 60/239,874. This application is also related to U.S. application Ser. No. 09/032,377, filed Feb. 27, 1998, and to U.S. Provisional Application Serial No. 60/039,564 filed Feb. 28, 1997. The disclosures of these applications are incorporated by reference in their entirety.

### FIELD OF THE INVENTION

The present invention relates to nanoparticles that display polyvalent binding units, each comprised of two or more different ligands, particularly where a first ligand binds or otherwise specifically interacts with a receptor on a target cell or substrate primarily based on structure, and a second ligand also specifically interacts with the same or a different receptor primarily based on charge or hydrophobicity. Such specific interactions occur under physiologically relevant shear conditions. The present invention specifically provides methods and composition for use in identifying, diagnosing, treating and preventing a variety of pathological conditions.

### BACKGROUND OF THE INVENTION

Nanoparticle-based therapeutics are important new forms of drugs and drug delivery systems for numerous reasons. The presentation of multivalent and polyvalent binding epitopes on the nanoparticle surface dramatically increases the avidity of the assembly for a receptor protein (or other receptor site) of interest (creating, for example, a Velcro®-like binding effect).

Also, dissimilar biological and/or chemical entities that bind to proximal binding sites on a receptor protein or carbohydrate (or other) moiety can be displayed on the same nanoparticle. Moreover, changing the size characteristic of the binding molecule (for example, by attachment to a macromolecule surface) can favorably alter the serum circulation half-life. Additionally, the hollow interior of many nanoparticles, including liposomes such as polymerized liposomes, can be used to deliver a payload (for example, therapeutic molecules, imaging agents or polynucleotides) to the cells or tissues of interest. Additionally, the release rate of such entrapped drugs or other payloads can be modulated, for example, by varying the degree of polymerization of a liposome or by other means of altering the “leakyness” of the nanoparticle.

The ligands, to which one wishes to raise an immune response can be polyvalently displayed, enhancing the immune response of a treated host (vaccine therapies). Appropriate ligands, include, for example proteins, peptides, antibodies, carbohydrates, nucleic acids, small organic molecules and mixtures thereof. Also, T-cell activating molecules can be co-displayed with vaccine target ligands enhancing a certain desired immune response. A variety of diseases and disorders may be treated by such nanoparticle constructs or assemblies, including: inflammatory diseases, infectious diseases, cancer, genetic disorders, organ transplant rejection, autoimmune diseases and immunological disorders. And peptides “hits” arising from the panning of a phage display library can be reconstituted in multivalent form to increase their activity. Along with the therapeutic aspects of these materials, nanoparticles displaying combinatorial libraries of different test ligands can be panned against known receptor(s) to discover new binding substances.

Multivalent Carriers and Polymerized Lipid Nanoparticles

Numerous multivalent constructs have been described in the literature. For example, it is known that some receptor binding sites contain relatively shallow binding grooves and their binding sites are solvent exposed. See, e.g., Kiessling et al., “Strength in Numbers: Non-natural Polyvalent Carbohydrate Derivatives,” Chemistry and Biology 3(2):71-77 (1996). Thus, for example, protein ligands make a relatively small number of direct contacts with their target ligands and this results in low discrimination, low affinity binding events. These authors describe the preparation of molecules bearing multiple carbohydrate residues, for example, three lactose residues attached to a scaffold of (6aminohexanamido-tris(hydroxymethyl)-methane). An increased binding affinity of 5-50-fold in a cell assay is reported. This article also describes the use of dendrimers for polyvalent carbohydrate display.

It is also well known that many biological systems interact through multiple simultaneous molecular contacts. See, e.g., a comprehensive review by Mammen et al., “Polyvalent Interactions in Biological Systems: Implications for Design and Use of Multivalent Ligands and Inhibitors,” Angew. Chem. Int. Ed. 37:2754-2794 (1998). These authors describe a wide variety of polyvalent reagents and the binding interactions between such reagents and various targets.

U.S. Pat. No. 5,702,727 to Amkraut et al., “Compositions and Methods for the Oral Delivery of Active Agents” (1997) describes compositions and methods for the oral administration of drugs and other active agents. Generally, the compositions comprise an active agent carrier particle attached to a binding moiety which binds specifically to a target molecule present on the surface of a mammalian enterocyte that promotes endocytosis or phagocytosis. The binding moiety is a composition that binds to the target molecule with a binding affinity or avidity sufficient to initiate endocytosis or phagocytosis of the particulate active agent carrier so that the carrier will be absorbed by the enterocyte.

Further according to the Amkraut et al. '727 patent, the carrier particle comprises a protective matrix that is suitable for encapsulating or otherwise retaining for example, by absorption or dispersion, an active agent. The active agent is thereafter released from the carrier into the host's systemic circulation. In this way, the degradation of degradation-sensitive drugs, such as polypeptides, in the intestines of a treated patient can be avoided while absorption of proteins and polypeptides from the intestinal tract is increased. The disclosed particulate drug carrier particles are said to include at least one binding moiety, and often from 10² to 10⁵ binding moieties in total. Such multivalency of the binding moiety is further said to increase binding avidity of the particles to the enterocyte target molecules, thereby increasing the binding avidity.

Multivalent liposomes, including polymerized liposomes, show enhanced binding of particles displaying multiple copies of an enkephalin unit. Imanishi et al., “Multivalent Ligands for Inducing Receptor-Receptor Interactions,” Pure Applied Chem. A31(11):1519-1533 (1994). These authors noted that enkephalin/lipid conjugates immobilized on the surface of polymerized lipid membrane surfaces showed lower affinities to certain classes of receptor (mu and delta receptors) than did free enkephalin. This was ascribed to nonspecific binding of polymerized liposomes to the bovine brain homogenate membrane, and attributed to hydrophobic interactions, expected to be overcome by increasing the hydrophillicity of the polymerized liposome. This was accomplished by adding or increasing the molar ratios of anionic lipids prior to polymerization. As reported, delta receptor affinity increased with an increasing content of anionic lipid in the polymerized liposome while the mu receptor affinity decreased.

Such polymerized nanoparticles have been described in various other patent and journal publications. For example, U.S. Pat. No. 6,004,534 to Langer et al., “Targeted Polymerized Liposomes for Improved Drug Delivery” (1999), relates to targeted polymerized liposomes for oral and/or mucosal delivery of vaccines, allergens and therapeutics. In particular, this patent describes polymerized liposomes that have been modified on their surface to contain a molecule or moiety that targets the polymerized liposome to a specific site or cell type in order to optimize uptake and the immune response to the encapsulated antigen or the efficacy of the encapsulated drug. In one disclosed embodiment, the polymerized liposomes are modified with lectins and targeted to the mucosal epithelium of the small intestine where they are absorbed into the systemic circulation and lymphatic circulation. Various methods for preparing and constructing polymerized liposomes also are described in this patent.

Polyvalent Nanoparticles

Prior to the present invention, various references also have described the use of different ligands simultaneously (thus, polyvalently) displayed on various carriers. Specifically with respect to polymerized nanoparticle, such carriers were reported to make effective binding agents to various receptors and targets, inhibiting biological interactions such as influenza virus binding to cells and selectin mediated cell recruitment.

For example, U.S. Pat. No. 5,962,422 to Nagy et al., “Inhibition of Selectin Binding” (1999), describes nanoparticles that are intended to provide a stable scaffold from which to present multiple ligands, particularly as required for P- and L-selectin inhibitors. Disclosed nanoparticles comprise a multivalent assembly of carbohydrates, interspersed with lipids bearing negatively charged head groups that provide for a high affinity, inhibitory activity. As described, these compositions are useful in inhibiting various biological phenomena mediated by selectins, including the adherence and extravasation of neutrophils and monocytes, and the trafficking of lymphocytes through blood vessels, lymphatics, and diseased tissue.

The Nagy et al. '422 patent further describes various methods that result in the inhibition of binding between a first cell having a P- or L-selectin and a second cell having a ligand for the selectin. In a preferred method, a lipid composition is permitted to interact with the first cell; wherein a proportion of the lipids are covalently crosslinked, a proportion of the lipids have an attached saccharide, and a proportion of the lipids not having an attached saccharide have an acid group that is negatively charged at neutral pH and which meets the anionic binding requirement of P- or L-selection. A proportion of the lipids having the attached saccharide or the acid group may be covalently crosslinked to other lipids in the construct, and a proportion may not be covalently crosslinked to other lipids.

In preferred embodiments of such Nagy et al. nanoparticles, a proportion of the lipids in the lipid construct have a first attached saccharide, and a separate proportion of the lipids have a second attached saccharide that is different from the first. It is suggested that the composition preferably has a 50% inhibition concentration (IC50) that is 10²-fold or 10⁴-fold lower than that of monomer sLe<x>. However, The inhibitory activity was remarkably high. In the cell bioassay, the sLe<x > analog-anionic lipid combination had an IC50 as low as 2 nM, which is up to 10⁶-fold lower than sLe<x > monomer. The lactose anionic lipid combination was effective at 15 nM. One benefit from such particles is that an effective therapeutic dose can be prepared at a lower cost and administered in a smaller volume than prior art compositions.

Also generally described by Nagy et al. are methods of inhibiting leukocyte adhesion or migration; methods for inhibiting leukocyte adherence or fibrin deposition; methods of inhibiting leukocyte adhesion or migration, methods of inhibiting lymphocyte adhesion, and other types of interventions in cell interaction mediated by selecting, comprising inhibiting binding between a first cell having a P- or L-selectin and a second cell having a ligand for the selectin.

Published PCT Applications No. WO 98/46270 of Advanced Medicine, Inc., “Molecules Presenting a Multitude of Active Moieties” (1998), discloses a composition that comprises a framework having multiple functional groups displayed thereon, where the functional groups may be attached to the framework via a linker. While the application mentions that polymerized liposomes may be used as the framework, the specific examples describe other types of polymer frameworks, such as polyacrylic acids. In general, this reference also describes the use of ancillary groups, including various charged molecules, to enhance the rigidity of the framework by encouraging the formation of certain conformations. With respect to liposomes, the use of charge is suggested to orient the “polyvalent presenter” (or ligand) with respect to the hydrophilic lipid framework. In addition, the use of shear flow assays to screen polyvalent presenters for useful properties is mentioned.

Published PCT Application No. WO/9847002, also of Advanced Medicine, “Polyvalent Presenter Combinatorial Libraries and Their Uses,” (1998), discloses the combinatorial use of frameworks bearing derivatives of poly(acrylic)acid-presenting sialosides as side chains as polyvalent inhibitors. Specific reference is made to inhibitors of influenza-mediated hemagglutination. The application further suggests that such polyvalent presenters may be used to identify compounds that inhibit cell-cell interactions, including selectin-mediated attachment of leukocytes to endothelial cells.

In addition, published PCT Application No. WO 99/64036 of Advanced Medicine, Inc., “Novel Therapeutic Agents for Macromolecular Structures” (1999), relates to “multibinding” agents. Such agents comprise a plurality of ligands, which can be the same or different, and each of which can bind to a macromolecular structure, for example, as may be found on a target cell. Such multibinding compounds or agents are defined as having 2 to 10 macromolecular ligands covalently bound to one or more linkers that may be the same or different. Such multivalency provides an increased biological or therapeutic effect, such as increased affinity, increased selectivity for target, decreased toxicity and improved bioavailability. A variety of linkers and suitable macromolecular ligands are suggested.

Similarly, U.S. Pat. No. 6,090,408 to Li et al., “Use of Polymerized Lipid Diagnostic Agents” (2000), relates to polymerized liposomes that are linked to a targeting agent, and that also may be linked to at least one of an image contrast enhancement agent and a therapeutic or treatment agent. In particular, this reference l0 discloses that the polymerized liposomes can be a mixture of lipids which provide different functional groups on the hydrophilic exposed surface. The functional surface groups may be groups such biotin, carboxylic acids, and others, which allow for attachment of targeting agents such as antibodies. The '408 patent also discloses the use of chelating functional groups such as diethylenetriamine pentaacetic acid for coupling a metal which provides for the paramagnetism and magnetic resonance contrast properties or for chelation of radioactive isotopes or other imaging agents.

U.S. Pat. No. 5,508,387 to Tang et al. (1996), relates to glyco-amino acid or glycopeptide compounds that bind to certain selectins and have selectin ligand activity. The glyco-amino acids and the glycopeptides have a three-dimensionally stable configuration for the presentation of a charged group, such as a carboxylic acid or a sulfate group, and a fucose group or analog or derivative thereof, such that the fucose group is covalently linked to an amino acid or peptide via a free carboxylic acid group, and such that the orientation of the charged group and the fucose group facilitates the binding of those groups to certain selectins. The '387 patent notes that the compounds of the invention may be reacted with suitably protected hydrophobic carriers such as ceramides, steroids, diglycerides or phospholipids to form molecules that act as immunomodulators. Moreover, the patent discloses that the compounds may be administered as injectables, with the active ingredient being encapsulated in liposome vehicles.

### SUMMARY OF THE INVENTION

The present invention is based, in part, on the discovery that multimerizing ligands on a nanoparticle surface, so as to produce polyvalent binding units, increases the avidity of the ligands by orders of magnitude. The invention expands upon prior research involving the creation of polyvalent inhibitors for selecting. Based on these observations, the present invention provides compositions and methods for use in various pharmaceutical and other applications.

In a preferred embodiment, the present invention relates to a nanoparticle that comprises a carrier, and polymerized liposome carriers are preferred, although various other carriers known to persons skilled in the art also would be appropriate. The carrier preferably carries or displays a first ligand and a second ligand, that is different than the first ligand. According to the methods and compositions of the present invention, this first ligand and second ligand form a polyvalent binding unit that is effective to produce a specific interaction between the nanoparticle and one or more receptors on a target under physiologically relevant shear conditions. Furthermore, the second ligand interacts specifically with said one or more receptors on the target based on its charge or hydrophobicity.

In a preferred embodiment, the receptor with which the polyvalent nanoparticles of the present invention interact is not a selectin. In another preferred embodiment, the ligands on the nanoparticle are not saccharides or similar structures.

### BRIEF DESCRIPTION OF THE DRAWINGS

FIG. 1 shows inhibition of P-selectin binding to a leukocyte model in the presence of nanoparticles according to the present invention.

FIG. 2 shows that administration of nanoparticles according to the present invention protects mice when challenged with C. albicans.

FIG. 3 shows inhibition of lymphocyte attachment in a Peyer's patch by administering nanoparticles according to the present invention in a shear assay.

FIG. 4 shows inhibition of neutrophil attachment in mouse mesentery venules.

FIG. 5 shows inhibition of neutrophil attachment in mouse ear venules.

FIG. 6 shows the orientation of a first ligand displayed on a nanoparticle-coated bead.

### DETAILED DESCRIPTION OF THE INVENTION

I. General Description

As ligands for cell receptors or pathogen attachment sites on cells are being discovered, new ways of delivering drugs or inhibiting pathogens can be developed from them. Many of the important ligands expressed on cells are carbohydrate in nature and generally do not individually bind with high affinity to their targets. In quite a few cases, the binding of a single carbohydrate to a single carbohydrate binding protein (a lectin) is quite weak. Thus, to exploit lectin ligands effectively as components of products for therapeutic intervention, high affinity binding moieties must be developed from such ligands. Since there are many copies of a carbohydrate receptor presented on the surface of a mammalian cell surface and, in a number of cases, there are also many copies of the lectin on the pathogen, this multi-point attachment leads to a tight interaction akin to a “Velcro-like” binding. This multipoint scenario also applies to toxins produced by pathogens. The present invention is based upon discoveries relating to compositions and methods for enhancing such a multi-point attachment binding scenario.

For example, macromolecules (in the form of nanoparticles), that bear similarly arrayed carbohydrate structures, can act as inhibitors of this kind of interaction when they are presented effectively to their corresponding protein receptor. Such constructs may be prepared from carbohydrate monomers and “matrix” or filler monomers mixed together in precise ratios and polymerized into spheroidal assemblies, on the nanometer size scale. Preferably, the carbohydrate, for example, is attached via a tether group to a lipid moiety to form the “tethered ligand monomers.” Other lipids fill the role of “filler” or “matrix monomers,” to which no ligands are attached or tethered. Preferably, these lipids are polymerized, according to techniques known in the art, in order to provide stability and a certain rigidity to the constructs.

The preferred embodiments of the present invention utilize this general approach to constructing nanoparticles, but additionally involve steps and components to substantially enhance binding affinity. Thus, the present invention relates to discoveries by the inventors that exploit not only the optimal percentage of tethered ligand monomers to matrix monomers, but also charge (positive or negative) or lack of charge on the nanoparticle. However, the present invention goes beyond simply adjusting the net charge (or zeta potential) of the nanoparticles. Specifically, it provides for the incorporation of an appropriate amount of “tethered charged group monomers” (or “charged head group monomers”) into the nanoparticles.

Accordingly, in the nanoparticle constructs of the present invention, a first or binding ligand is displayed in a matrix or lipid monomers displaying a second ligand. These second ligands are selected from groups that, at physiologic pH, are charged or neutral and which are covalently linked via a tether (or other linker moiety) to a lipid monomer. Preferred charged head group may be acidic (e.g., using carboxylic acid, sulfate or phosphate groups, etc.), neutral (e.g., using hydroxyl groups) or basic (e.g., using amine groups). The interaction of such first and second ligands creates polyvalent binding units that optimize the binding of the nanoparticles to their receptor(s) on a variety of target tissues and substrates.

The enhanced binding affinity of such nanoparticles provides an enhanced delivery vehicle for various therapeutic compositions. As is well known in the art, non-polymerized nanoparticles, such as liposomes, have been used to change the pharmacodynamics of therapeutic substances either entrapped inside their structures or displayed on their surfaces. The macromolecular nature of the assemblies covered with surface targeting ligands can, in some cases, retard some of the physiological pathways, generally enzymatic, that when activated would ordinarily degrade such ligands. The present invention similarly proposes to make use of this property, especially with highly sensitive drug ligands such as carbohydrates, peptides, proteins and genetic material (DNA, RNA, etc.). In addition, polymerizing the bilayer structure makes the assembly dramatically more resistant to digestive breakdown in the stomach compared to conventional, phosphotidylcholine-based liposomes.

Entrapment of sensitive or toxic molecules within the nanoparticle can shield the material from degradative processes or immuno-recognition. This is an important aspect of the present invention when considered in its drug delivery embodiments. The demonstration of this principal has been described and is known in the art with regard to conventional bilayer liposomes. The escape rate of the entrapped drug is largely controlled by the lipophilicity of the drug or its solubility in the lipid membrane. Hollow, polymerized nanoparticles, on the other hand, can be formulated with a defined “leakyness” by having pores of an optimal size. In this way, engineering the entrapping nanoparticle can modulate the optimal escape rate of any drug, and techniques to modulate leakyness and escape or release rates also are known in the art.

In general, the polymerized nanoparticle constructs and assemblies of the present invention have utility as modulators, inhibitors and enhancers and drug delivery agents for a variety of interactions as well as their down-stream effects, such as pathogen-cell attachment, pathogen-derived-toxin-cell attachment, cell-cell attachment mediated diseases, integrin adhesions, complement fixation and chemokine-mediated events.

Polymerized nanoparticles can be readily used as synthetic vaccines. As noted above, individual “small” ligands, especially carbohydrates, are difficult to administer and generally fail to elicit an effective immune response. Thus, combining multiple copies into a polyvalent display with the polyvalent binding units of the present invention would enhance the immuno-recognition by a vaccinated host, particularly human beings and commercially important livestock and other animals. In addition, such polyvalent binding units can be displayed along with immunogenic peptides to direct the nanoparticle to the appropriate immune cell, such as the tetanus toxoid antigen to the T or B-cell or the sigma factor to the M-cell. From these nanoparticle vaccines we can expect highly intense, anamnestic and long-lasting immune responses (several years). In this way, the multivalency and high local concentrations of epitopes which favor the formation of high-affinity complexes required to activate B cells are coupled with the Th/B cell collaboration needed for optimal induction of the antibody response.

Phage display library technology is currently being utilized to discover many interesting peptide ligands. A severe limitation of that technology is in recreating the binding activity of the identified peptide while it is unattached to the phage arms. In many cases, the single peptide is simply unable to reproduce the three-dimensional architecture that was present on the pentavalent phage display. However, reassembling them in polyvalent form, for example, on polymerized nanoparticles, often can restore the immunological activity of such peptides that have been isolated from the phage library.

Additionally, the nanoparticles of the present invention may be used in conjunction with combinatorial libraries to display binding epitopes. Using traditional combinatorial library synthesis, nanoparticles displaying a very large variety of ligands can be prepared. By analogy to the conventional “one-bead-one-analog” library approach, the nanoparticles can have “one-nanoparticle-one-analog” displayed polyvalently on its surface. The population of nanoparticles is exposed to the receptor of interest and an assay is conducted to see if any bind. Ligand displaying nanoparticles can be used in much the same way as phage display libraries.

A key difference is that the nanoparticles cannot reproduce themselves as phage can. This difference is significant, thus making the isolation of a single nanoparticle “hit” from the non-binding population a daunting task. However, if a collection of visually removable polymer beads are used each as a carrier of a population of unique nanoparticles, the task of identifying the surface epitope is enormously simplified. In general, a polymer bead (about 100 microns in diameter) is covered with nanoparticles each polyvalently displaying a unique epitope on their surface according to the present invention) and is exposed to a receptor. If a binding occurs, the entire bead is identified and physically removed for analysis. This allows for the creation of precise arrays of binding epitopes on nanoparticles to be coupled with the ease of manipulation of visual beads.

II. Definitions

As used herein, the term “attachment adhesion” means the process by which a cell, such as a leukocyte, for example, having formed an attachment adhesion to a substrate, such as an endothelial vessel wall, arrests its motion via attachment to receptor(s) on that surface. Typically, attachment adhesion is mediated by integrins and involves the sticking and flattening of adherent cells.

As used herein, the terms “displayed” or “surface exposed” are considered to be synonyms, and refer to molecules that are present (e.g., accessible to receptor/ligand interactions) at the external surface of a structure such as a nanoparticle.

As used herein, “head groups” or “end groups” refers to molecules that are attached via a tether or linker to a nanoparticle and which form specific binding interactions with receptor(s) on a target. Such head groups may be charged, hydrophobic or polar (hydrophilic). For example, a negatively charged head group is comprised of an lo acidic group, a sulfate group, or a phosphate group, that is negatively charged at physiological pH, while a positively charged head group may comprise a basic group, such as an amine, that is positively charged at neutral pH. Hydrophobicity may be imparted through the use of hydrophobic groups, such as aliphatic hydrocarbons or aromatic rings. Preferred head groups include carbohydrates.

As used herein, the term “ligand” means any ion, molecule, molecular group, or other substance that specifically interacts with (and, preferably, binds to) another entity (that is, a receptor) to form a larger complex. Examples of ligands include, but are not limited to, peptides, carbohydrates, nucleic acids, antibodies or any molecules that specifically interact with and/or bind to receptors. It is generally preferably to utilize ligand that are readily attached to a nanoparticle via a linker molecule that retains an effective level of interaction or binding affinity following linkage.

As used herein, the terms “linker” or “spacer” means the chemical groups that are interposed between the nanoparticle and the ligands. Preferably, the linkers are covalently attached to the ligands and one end and at their other end to the nanoparticle.

As used herein, the term “liposome” is defined as an aqueous compartment enclosed by a lipid bilayer. (Stryer, Biochemistry, 2d Edition, W. H. Freeman & Co., p. 213 (1981)). In general, liposomes can be prepared by a thin film hydration technique followed by a few freeze-thaw cycles. Liposomal suspensions can also be prepared according to methods known to those skilled in the art, for example, as described in U.S. Pat. No. 4,522,811. Polymerized liposomes may be prepared, for example, as described in U.S. Pat. No. 5,962,422.

As used herein, the term “nanoparticle” means a polymer sphere or spheroid that can be formulated to have a regular arrayed surface of defined, tethered molecules in the nanometer size range (about 20 nm to 500 nm). Preferably, self-assembling monomers are utilized to form the nanoparticles. Moreover, the term nanoparticle encompasses the use of both polymerized and unpolymerized liposomes, bicelles and micelles, as well as viral capsid structures. Although nanoparticles are preferred for the compositions and methods of the present invention, other frameworks, scaffolds and other “presenters” such as dendrimers may be used as would be well known to persons skilled in the art as being appropriate to present ligands according to the present invention.

As used herein, the term “nonnaturally occurring peptides” means peptides that incorporate an amino acid which is not one of the 20 naturally occurring amino acids.

As used herein, the term “pathogenic protein” proteins that mediate or are associated with various disease conditions, such as the amyloid protein as deposited in Amyloidosis and prion proteins.

As used herein, “physiologically relevant shear conditions” means those shear conditions that correspond to the shear forces in living organisms in the intestinal tract, mucosal tract, pulmonary system and circulatory system. In general, shear forces of approximately about 0.1 to 10 dynes per square centimeter are contemplated and preferably about 1-2 dynes per square centimeter are contemplated.

As used herein, the term “polymerized” or “polymerization” encompasss any process that results in the conversion of small molecular monomers into larger molecules consisting of repeated units. Typically, polymerization involves chemical crosslinking of molecular monomers to one another.

As used herein, the term “polymerized liposome” means a liposome in which the constituent lipids are covalently bonded to each other by intermolecular interactions. The lipids can be bound together within a single layer of the lipid bilayer (the leaflets) and/or bound together between the two layers of the bilayer.

The degree of crosslinking in the polymerized liposomes preferably ranges from about 30 to 100 percent, that is, up to I100 percent of the available bonds are made. The size range of polymerized liposomes preferably is between about 20 nmn to 500 nm in diameter, preferably less than about 200 nm and more preferably less than about 100 mnm. As is well known to persons skilled in the art, liposomes may be loaded with a wide variety of agents. Liposomes: Rational Design, ed. A. S. Janoff (1999), Marcel Decker, publ.

As used herein, the term “polyvalent” means that more than one type or class of ligand molecule are displayed on a nanoparticle, preferably via tethers attached to component monomers. Moreover, the one or more types or classes of ligand molecules may be attached to the nanoparticle through two separate tethers, or may be attached to the nanoparticle via a common tether.

As used herein, the term “polyvalent binding units” means two (or more) ligands that collectively contribute to the specific interactions, such as binding, between the nanoparticle and the receptor(s) with which it specifically interacts.

As used herein, the term “antigen processing receptor” refers to receptors that mediate the uptake and processing of antigens, and then present the antigens for the development of immunity. Such receptors may be found on, for example, M-cells, dendritic cells and macrophages.

As used herein, the term “ProteoFlow Index” or “PFI”, is a description of the effect that a given nanop article may have on the binding interactions of a cell to its native target cell or tissue. The PFI can be derived experimentally using an in vitro shear assay system as described, for example, in Bargatze et al., J. Immunology 152:5814-5825 (1994), and maybe expressed as:

PFI (rolling/sticking)=% reduction in rolling sticking cells over the control divided by the ratio of:

$$\frac{µg ( total nanoparticle polymer weight )}{ml ( total blood volume )}$$

PFI (cell velocity)=% increase in velocity of cells over the control divided by the ratio of:

$$\frac{µg ( total nanoparticle polymer weight )}{ml ( total blood volume )}$$

As used herein, the term “rolling adhesion” means the process by which a cell, such as a leukocyte, begins to form an attachment via specific binding interactions with a surface such as an endothelial vessel wall. Typically, the relevant endothelial cell receptors involved in rolling adhesion are integrins and selecting.

As used herein, the term “specific binding interaction” means an interaction between ligands and one or more receptors based on complimentary three dimensional structures and/or charge or hydrophobicity.

III. Specific Embodiments

In general, polyvalent polymerized lipid compositions of the present invention are produced according to techniques described in Nagy et al., U.S. Pat. No. 5,962,422, discussed above, utilizing the materials and methods disclosed therein. Additional materials and methods contemplated for the present invention are described in U.S. application Ser. No. 09/032,377, filed Feb. 27, 1998, and in U.S. Provisional Application Serial No 60/039,564 filed Feb. 28, 1997.

In general, it will be readily appreciated that the practice of this invention is not critically dependent on the chemical details of the composition. Within the constraints of the three requirements above, the practitioner is free to assemble the composition according to a number of different approaches. Variations in polymerization chemistry and the conjugation of determinants are permitted and included in the scope of this invention. Designing particular linkages between ligands and lipid monomers also is well within the skill of the ordinary practitioner. The optimization of such linkages and compounds may achieved by routine adjustment and following the effects of adjustment on receptor binding in one of many assays established in the art.

In general, when assembling nanoparticles according to the present invention, a certain proportion of the lipids in the nanoparticle are attached to the first ligand, and a distinct proportion of the lipids in the nanoparticle are attached to the second ligand that is different from the first ligand. It is important to note that the first and second ligands are displayed randomly on the nanoparticle. In effect, the receptor(s) accept those polyvalent binding units formed by first and second ligand pairs that have the optimal spacing and charge/hydrophobicity characteristics. The preferred embodiments of the invention are produced according to the methods described herein, in which the relative amounts and respective ratios of the monomers bearing fist ligands and seconds ligand as well as filler monomers are determined empirically.

While it is not critical that particular first and second ligands always be chosen with respect to particular receptors, it is important that the first ligand specifically interacts with (or binds to) that receptor, and that the first and second ligands together are capable of forming a polyvalent binding unit having enhanced binding characteristics with respect to that receptor(s). When a third (or additional) ligands are utilized in the nanoparticles of the present invention, the three separate ligands also must be capable of forming a polyvalent binding unit having such enhanced binding characteristics with respect to that receptor(s) on the target. Exemplary ligand pairs (and triplets) are described in the attached Table 1.

TABLE 1
Exemplary Receptors and Polyvalent Binding Ligands:

| RECEPTOR | Ligand 1 | Ligand 2 | Ligand 3 |
|---|---|---|---|
| 1) selectin proteins | sulfo or sialyl | anionic grps | |
| | Lewis X | (sulfates) | |
| | glycomimetics | | |
| 2) selectin binding | EL-246 peptide | cationic grps | |
| carbohydrates (found | | (amines) | |
| on leukocytes) | | | |
| 3) selectin proteins, | sLex, SO3-Lex | anionic grps | integrin |
| integrin proteins | glycomimetics | (sulfates) | ligands |
| (RGD) | | | |
| 4) selectin binding | EL-246 peptide | cationic grps | integrin |
| carbohydrates, | | (amines) | ligands |
| (RGD) | | | |
| integrin proteins | | | |
| 5) verotoxin | galactose β1-4 | cationic grps | |
| | galactose | (amines) | |
| 6) Antigen Processing | Candida Albicans | anionic grps | |
| Receptor | cell wall extract | (carboxylic acids) | |
| | (glycoprotein) | | |
| 7) Antigen Processing | Candida Albicans | anionic grps | T-cell |
| Receptor | cell wall | (carboxylic acids) | directing |
| | oligosaccharides | | peptides |
| | | | (Thepitope) |
| 8) M-cell | sigma protein | anionic grps | |
| | | (carboxylic acids) | |
| 9) M-ceII | sigma protein | anionic grps | Antigens, |
| | | (carboxylic acids) | DNA |
| 10) selectin proteins | fucopeptides | anionic grps | |
| | | (sulfates) | |
| 11) Antigen Processing | Candida Albicans | anionic grps | |
| Receptor | glycomimetic from | (carboxylic acids) | |
| | phage library | | |

The following description and examples are provided merely as an illustration of possible approaches and preferred embodiments. Persons skilled in the art will readily understand that various modifications may be made according to the teachings herein.

Examples of Preparation of Components of the Lipid Composition:

One embodiment of the present invention uses lipids both to bear the determinants required to inhibit selectin binding, and as components for forming the lipid assemblies. Examples of lipids that can be used in the invention are fatty acids, preferably containing from about 8 to 30 carbon atoms in a saturated, monounsaturated, or multiply unsaturated form; acylated derivatives of polyamino, polyhydroxy, or mixed aminohydroxy compounds; glycosylacylglycerols; phospholipids; phosphoglycerides; sphingolipids (including sphingomyelins and glycosphingolipids); steroids such as cholesterol; terpenes; prostaglandins; and non-saponifiable lipids.

When a negatively charged head group is utilized as the second ligand of a polyvalent binding unit, it is typically an acid accessible from the exterior surface of a nanoparticle. In certain embodiments, the acid is an organic acid, particularly a carboxylic acid. In other embodiments, the acid is an oxyacid of the form (XO[n])(O—)[p], wherein n+p>2. In this case, the lipid will typically be of the form R[m](XO[n])(O—)[p] wherein each R comprises an aliphatic hydrocarbon (which are not necessarily the same), m is 1 or 2, (XO[n])(O—)[p ]is an oxyacid, and n+p>2.

Preferred oxyacids are sulfate, SO3—, and phosphate. A phosphate may be conjugated through one or two of its oxygens to aliphatic hydrocarbons. For any negatively charged component of the composition, any additional features may be present between the acid and the aliphatic or membrane anchoring group. These include spacers such as polyethylene glycols and other heteroatom-containing hydrocarbons. The acid group may also be present on a substituent such as an anino acid, a sugar, or a pseudo-sugar, which includes phosphorylated or sulfated forms of cyclohexidine, particularly hexaphosphatidyl inositol and hexasulfatidyl inositol.

The negatively charged group may already be present in the lipid, or may be introduced by synthesis. Examples of lipids with negatively charged head groups include the fatty acids themselves (where the negative charge is provided by a carboxylate group), cardiolipin phosphate groups, dioleoylphosphatidic acid (phosphate groups), and the 1,4-dihexadecyl ester of sulfosuccinic acid (sulfate group).

Negatively charged lipids not commercially available can be synthesized by standard techniques. A few non-limiting illustrations follow. In one approach, fatty acids are activated with N-hydroxysuccinimide (NHS) and 1-(3-dimethylaminopropyl)-3-ethylcarbodiimide (EDC) in methylene chloride. The leaving group N-hydroxysuccinimide can be displaced with a wide range of nucleophiles. In one example, glycine is used to yield a fatty acid-amino acid conjugate with a negatively charged head group. Glutamic acid can be coupled to the activated fatty acid to yield a fatty acid-amino acid conjugate with two negative charges in its head group. In another synthetic approach, 2,3-bis((I-oxotetradecyl)oxy)-butanedioic acid is prepared by adding myristoyl chloride in toluene to a pyridine solution of dl-tartaric acid. The clarified solution is concentrated to yield the product, which is recrystallized from hexane (Kunitake et al., Bull. Chem. Soc. Japan, 51:1877, 1978).

A sulfated lipid, the 1,4-dihexadecyl ester of sulfosuccinic acid, is prepared as follows: a mixture of malice anhydride and hexadecyl alcohol in toluene with a few drops of concentrated sulfuric acid is heated with azeotropic removal of water for 3 h. The dihexadecyl maleate is recrystallized, then heated with an equimolar amount of NaHSO3 in water at 100° C. for 2-3 h. The product is recovered by evaporating the water and extracting the lipid into methanol (Unitake et al., supra). Alkyl sulfonates may be synthesized as follows. A lipid alcohol is obtained from Sigma, or the acid group of a fatty acid is reduced to an alcohol by reacting with lithium aluminum hydride in ether to convert the carboxylate into an alcohol. The alcohol can be converted into a bromide by reaction with triphenylphosphine and carbon tetrabromide in methylene chloride. The bromide is then reacted with bisulfite ion to yield the alkyl sulfonate.

Sulfates may be prepared by reacting an activated fatty acid with a sulfate-containing amine. For example, the N-hydroxysuccinimide ester of 10, 12-pentacosadiynoic acid is reacted with taurine to yield N-I0, 12-pentacosadiynoyl taurine. Sulfates may also be prepared by reacting an alcohol, e.g. lauryl alcohol, with sulfur trioxide-trimethylamine complex in anhydrous dimethylformamide for 2.5 h (Bertozzi et al., Biochemistry 34:14271, 1995).

Phosphate-containing lipids not commercially obtainable are also readily synthesized. For example, to prepare dialkyl phosphate compounds, phosphoryl chloride is reacted with the corresponding alcohol. To make dihexadecyl phosphate, phosphoryl chloride is refluxed with three equivalents of hexadecyl alcohol in benzene for twenty hours, followed by recrystallization of the product (Kunitake et al., supra). Monoalkyl phosphates may be prepared by reacting, e.g., 10, 12-hexacosadiyne-1-ol (1 eq.) with phosphoryl chloride (1.5 eq.) at ambient temperature in dry CC14 for approximately equal to 12 h, then boiling under reflux for 6 h. Removal of the solvent and heating the residue with water for 1 h yields the desired 10, 12-hexacosadiyne-1-phosphate (Hupfer et al., Chem. Phys. Lipids 33:355, 1983). Alternatively, a fatty acid activated with NHS can be reacted with 2-aminoethylphosphate to yield the acylated derivative of aminoethylphosphate.

Carbohydrate components suitable for use with this invention include any monosaccharides, disaccharides, and larger oligosaccharides appropriate binding activity when incorporated into a polymerized lipid carrier or nanoparticle. Simple disaccharides, for example, lactose and maltose, have no selectin binding activity as monomers, but when incorporated into polymerized liposomes acquire substantial activity. Accordingly, the range of suitable carbohydrates for selectins and other receptors(s) is considerable.

Exemplary first ligands and second ligands (and in some cases a third ligand) are identified in Table 1. With respect to those embodiments in which the first ligand is a carbohydrate, such carbohydrate may be a disaccharide or neutral saccharide with no detectable binding as an unconjugated monomer. In other embodiments, such carbohydrates have substantial binding in the monomeric form, and are optionally synthesized as a multimeric oligosaccharide, although this is not typically required. Preferred oligosaccharides are sialylated fucooligosaccharides, particularly sLe<a > and sLe<x>, analogs of sialylated fucooligosaccharides, sulfated fucooligosaccharide, particularly sulfo Le<x>, and analogs of sulfated fucooligosaccharide. Disaccharides and larger oligosaccharide may optionally comprise other features or spacer groups of a non-carbohydrate nature between saccharide units.

Also, generally, the preferred nanoparticle compositions of the present invention have an IC50 in the range of about 0.1 nM to 1 μM, and preferably in the range of about 1 nM to 100 nM under physiologically relevant shear conditions. Generally, binding specificities less than about 100 nM are preferred. This IC50 is based on a theoretical molecular weight of the nanoparticle being about 90 million daltons.

Preparation of Preferred Nanoparticles (PLNs):

Polymerized polydiacetylene (PDA) liposomes were prepared according to the method previously described. Spevak, et al., “Carbohydrates in an Acidic Multivalent Assembly: Nanomolar P-Selectin Inhibitors,” J. Med. Chem., 39:1018-1020 (1996). Briefly, polymerizable matrix lipids, neoglycolipids, peptidolipids, or charged lipid were mixed and evaporated to a thin film. Adequate mixing of matrix lipids is needed to ensure that the spacing between binding ligands is enough to allow the liposome to become polymerized. Typically 50% or more matrix lipid is sufficient to accomplish this requirement. Deionized water was added to the films so as to give a desired concentration of total lipid in suspension. The suspension was heated to between 70-80° C. and probe sonicated for 30 min. The resulting clear solution was then cooled to 5° C. for 20 min. and polymerized by LV light irradiation (254 nm). The deeply colored solutions were syringe filtered through either 0.8. 0.65, 0.45 or 0.2 μm filters in order to remove trace insoluble aggregates, metal or dust particles and any PLNs above a desired size range.

The carbohydrate content in several neoglycolipid containing PLN assemblies was assayed by FACER analysis with the Monosaccharide Composition Kit (Glyko, Inc., Novato, Calif.) or for total carbohydrate via Dionex assay. Peptide or protein displaying PLNs were assayed prior to polymerization for total protein concentration by a BCA protein quantification kit.

### EXAMPLES

### Example 1

Sialyl Lewis X Carbohydrates Polyvalently Displayed on PLN.

Sialyl Lewis X (sLex)-like carbohydrates (3′-acetic acid, 3-fucosyllactose; 3′-sulfo, 3-fucosyllactose, 3′-sialyl, 3-fucosyllactose, or fucose) are covalently attached through a linker of about 10-30 atoms to a polydiacetylene polymer backbone. The polymers self-assemble into spheres or spheroidal balls having a diameter in the range of about 10 to 250 nm. These spheres are formulated in a size range of about 20 to 150 nm. Generally, the end groups of the monomers on the outer surface of the particle that are substituted with a first ligand (here carbohydrate groups) are in the range of about 1 to 40% carbohydrate groups. The overall optimal substitution by carbohydrate of the outer surface of the nanoparticle generally is about 2 to 15%.

Additional end groups of the monomers on the outer surface of the nanoparticle's component polymers are substituted with a second ligand, preferably a chemical group that has an anionic charge at physiological pH (such as carboxylic acids, phosphates, sulfates or hydroxamic acids). Preferably, the substitution of end groups with such anionic molecules is in the range of about 5 to 60%, with an optimal range generally being about 15% to 35%.

The balance of the nanoparticle matrix is made up of hydrophilic but chemically neutral monomers.

The anionic groups provide a binding effective spatial charge distribution in the three-dimensional vicinity of carbohydrate (or other first ligand) moieties on the nanoparticle that serve the function of supplying a charge like that of the sulfated tyrosine residues in the neighboring peptide backbone to the sialyl Lewis X glycosylation site on the physiological ligand (PSGL-1), shown to be crucial in P and L-selectin recognition.

In order to measure the interaction or binding affinity of the nanoparticle constructs, in vitro and in vivo measurement may be made. For example, the nanoparticles are administered to an anesthetized mouse, where they inhibit the rolling and sticking of lymphocytes or leukocytes. This inhibition is measured in the vasculature of several tissues known to be models of selectin mediated cell recruitment (for example, activated skin, activated mesentery and Peyer's patch). The inhibitory activities of the nanoparticles are measured by a ProteoFiow apparatus to assess: (1) the per cent reduction in rolling and sticking cell vs. the control; and (2) the per cent increase in the velocity of cells that do interact with the endothelium test substrate vs. the control. A qualitative estimate of adhesion blocking ability, the ProteoFlow Index (PFI), can be derived for each measurement parameter and are expressed as:

$$\begin{matrix}PFI ( rolling / sticking ) = \frac{\% reduction in rolling sticking cells over the control}{\frac{µg ( total nanoparticle polymer weight )}{ml ( total blood volume )}} \\ PFI ( cell velocity ) = \frac{\% increase in velocity of cells over the control}{\frac{µg ( total nanoparticle polymer weight )}{ml ( total blood volume )}}\end{matrix}$$

Preferably, nanoparticle selectin inhibitors should have PFI indices in the range of about 0.5 to 50 for optimal effectiveness. Persons skilled in the art will understand that other measurements may be made by various techniques, such as ELISA assays.

### Example 2

Synthetic Selectin-Like Binding Site Peptide (EL-246) Displayed Polyvalently on PLN.

Similar to the sialyl Lewis X selectin blocking PLN described in Example 1, above, nanoparticles are constructed to display the peptide epitope identified through a phage library panning against EL-246 antibodies. The peptide epitope is a synthetic mimetic of the carbohydrate-binding domain of E and L-selectin. Therefore, the nanoparticles displaying this epitope bind to the carbohydrate selectin ligands (e.g., sialyl Lewis X) and block their recognition by selectin. There are sulfated tyrosine residues near the glycosylation sites on the physiological L-selectin ligand or sulfate groups on the carbohydrate itself. Inclusion of cationic groups on the EL-246 peptide-displaying PLN enhances the binding of the assemblies to neutrophils, likely through a charge attraction to the aforementioned anionic sulfated sites. Specifically, the necessary percentage of peptide is approx. 20% of the surface and the necessary amine group coverage is about 40%. In addition to the sialyl Lewis X selectin blocking PLN, this example further serves to demonstrate that auxiliary binding sites in the form of charged residues in proximity to the primary binding site allows for the construction of very specific PLN constructs with high binding activity. Nanoparticle selectin inhibitors have PFI indices in the range of 0.5 to 50.

### Example 3

Synthetic Selectin Binding Carbohydrate Displayed Polyvalently on the Surface of Unpolymerized Liposomes.

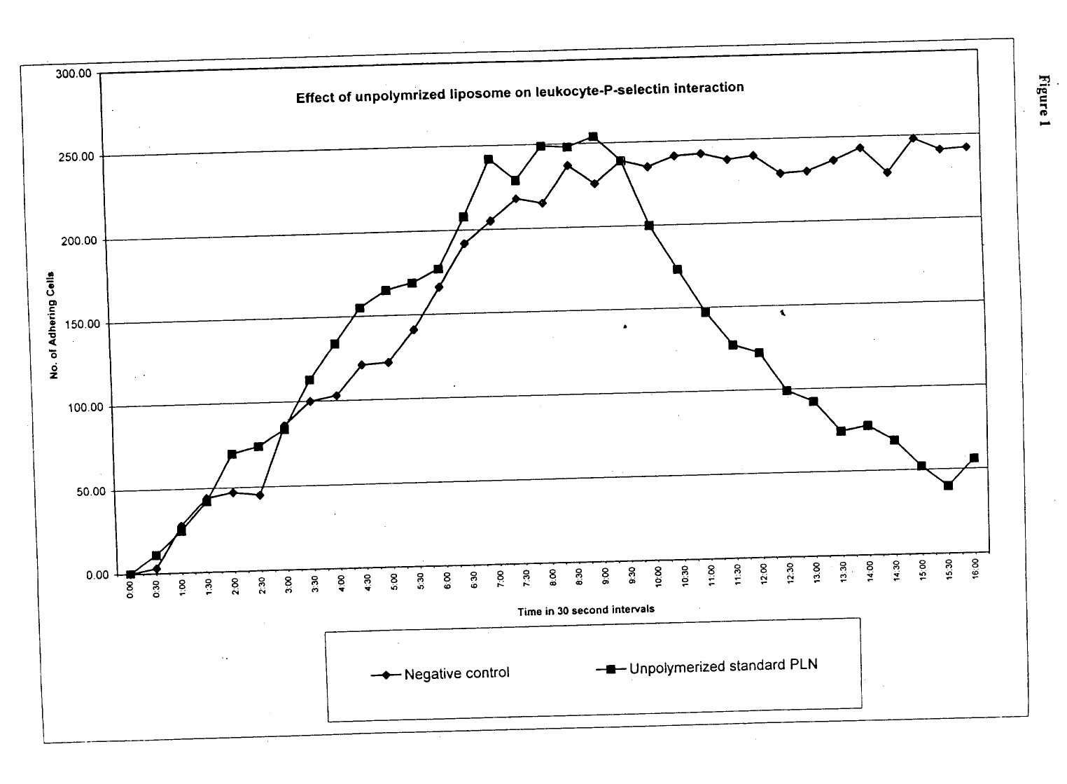

Similar to the selectin blocking nanoparticles described in Examples 1 and 2, above, nanoparticles of unpolymerized lipid monomers are constructed to display a selectin-binding carbohydrate. The nanoparticle displaying this carbohydrate binds to P-selectin, found on endothelial cells and platelets. Inclusion of cationic groups on the carbohydrate-displaying unpolymerized liposome enhances binding of the liposome-bound carbohydrate to P-selectin, thus competitively inhibiting the binding of leukocytes to cells expressing P-selectin on their surface. In FIG. 1, the negative control data shows U937 myeloid cell (a common leukocyte model) binding to P-selectin in the absence of the nanoparticle; the “unpolymerized standard” shows the effects of leukocyte adhesion following injection of the nanoparticle at t=9 minutes, with the nanoparticle decreasing leukocyte/P-selectin interactions by greater than 75%.

### Example 4

Sialyl Lewis X Carbohydrates or EL-246 Peptides Polyvalently Displayed on Stealthed PLN.

The removal of circulating nanoparticles by sequestration into phagocytic cells would be expected to have a deleterious effect on the drug potency. Except for the possibly beneficial slow release of nanoparticles back into solution from the “RES reservoir,” thereby modulating the pharmacokinetics of a dose-related toxicity, this type of recognition generally can be minimized. The liposome field was advanced in this respect by the discovery of various “stealth” agents that coat the vesicle surface, thereby camouflaging the material from RES surveillance. Two of the most successful agents in this regard are polyethylene glycol (PEG) polymer chains and the complex oligosaccharide GM₁. These materials act as a steric barrier to recognition and binding of phagocytes. See, e.g., Bendas et al., “Selectins as New Targets for kinunoliposome-mediated Drug Delivery. A Potential Way of Anti-inflammatory Therapy,” Pharm. Acta Helv. 73(1):19-26 (1998).

The incorporation of PLN masking strategies with either a displayed Sialyl Lewis X carbohydrate or EL-246 peptide epitope has been evaluated. The extent of shielding of the binding epitope vs. shielding of the entire nanoparticle assembly from RES uptake was determined experimentally. At surface percentages 0.5 to 5% of PEGalated lipid the circulation half-life of the PLN is dramatically increased with minimal reduction in selectin inhibition activity by ProteoFlow shear assay analysis.

### Example 5

Dual Function PLN with Integrin Ligands Polyvalently Co-Displayed with Sialyl Lewis X Carbohydrates or EL-246 Peptides.

Similar to the sialyl Lewis X or EL-246 selectin blocking PLN described above, nanoparticles are constructed to display in addition to either sialyl Lewis X or EL-246 binding groups, the RGD peptide that is recognized by β-1 integrins. This bifunctional PLN is designed to block both the selectin-carbohydrate recognition (responsible for initial cell tethering and rolling) and integrin-peptide recognition (responsible for firm cell arrest on endothelia). In this way, the dual epitope binding to different cell surface molecules allows for an even more effective blockade of rolling/tethering/arrest of leukocytes that either group by itself. Specifically, the optimal surface percentage of sialyl Lewis X and RGD peptide are both approx. 5% in this assembly. In the EL-246 peptide/RGD peptide assembly the percentages are 20% and 5%, respectively. In the preferred nanoparticles according to the present invention, the sialyl Lewis X or EL-246 selectin blocking would be considered to be the first ligand, a charged head group would be the second ligand and the RGD peptide would be considered to be the third ligand. See, e.g., Table 1, above.

### Example 6

PLN with Verotoxin-Binding Carbohydrates Displayed Polyvalently.

Similar to the sialyl Lewis X selectin blocking PLNs described above, nanoparticles are constructed to display the carbohydrate epitope found on Daudi or Vero cells that is recognized by the verotoxin. Cytotoxic strains of E. Coli produce a toxic lectin that binds to the galactose β1-4 galactose disaccharide residues found on the target cell surface. The present inventors have found that PLNs displaying this disaccharide epitope can “soak up” this toxin, thereby rendering the resulting nanoparticle-toxin complex essentially non-reactive toward cells. At PLN-bound carbohydrate concentrations of about 40 μM substantially complete blocking of toxin binding to Daudi cells was achieved.

The demonstration that multivalent ligand recognition by verotoxin is key to activity allows the application of the PLN technology according to the present invention to vary the surface carbohydrate arrays to optimize toxin binding. By changing the percentage of carbohydrate to matrix groups, a person skilled in the art can optimize the ligand presentation to tailor fit it to the toxin lectin array.

A surprising observation made in the course of screening a panel of galactose β1-4 galactose PNL formulations with toxin and Daudi cells, showed that in this system the matrix charge (again) played a major role in binding affinity and toxin-neutralizing activity. By optimizing and then keeping the first ligand carbohydrate percentage constant and then varying the second ligand charge groups from basic to neutral to acidic, this study showed that only the amino (basic) second ligands led to an active formulation, which could be optimized experimentally by preparing and evaluating a progressive series of fornmulations having varying proportions of monomers to which such charged head groups were attached.

Such results are quite similar to those obtained with the selectin blocking PLN formulations. Thus, a similar charge pairing appears to be important in enhancing the binding affinity of the carbohydrate-binding domain of the toxin. A review of the key amino acids in the toxin binding sites indeed revealed the presence of essential acidic groups: aspartic acid 32, and multiple glutamic acid residues in positions 27, 30 and 130 (Maloney and Lingwood, J. Exp. Med. 1994, 180, 191-201). This strongly suggests that in addition to arrayed carbohydrate recognition the amino groups are participating in binding to the acidic residues, to greatly augment to overall binding potency of the PLN. Persons skilled in the art might optionally, for example, examine the amino acid sequences of extracellular domains of cell surface receptors to find at least an indication that the polyvalent binding unit approach of the present invention might be beneficial for a given receptor.

Specifically with respect to these nanoparticles, the carbohydrate coverage was 15% of the surface and the amine coverage was 85%. Again, the relative simplicity of the PLN technology allows a person skilled in the art to produce a progressively varying series nanoparticle constructs and to assay them in various systems.

### Example 7

PLN with Malaria-Binding Carbohydrates Displayed Polyvatently.

The invasion of erythrocytes involves specific interactions between parasite ligands and cell-surface receptors. If this invasion into human erythrocytes were inhibited, the malaria life cycle would be interrupted and the disease attenuated or prevented. Various studies by the present inventors have been initiated to understand at the molecular level the basis for erythrocyte invasion and further adhesion to endothelial cells.

The results of several studies suggest that carbohydrate (sialic acid) containing peptides (glycophorins) are the major components in pathogen recognition and binding to injectable red blood cells. In competitive binding studies, however, the presence of sialic acid alone is not sufficient to inhibit binding. It appears that a very special arrayed presentation of sialic acid or sialic acid containing oligosaccharides may be necessary for recognition, a situation not unlike that involved in selectin/sialyl Lewis^{X} recognition.

Several linear polymers containing sialic acid were synthesized and studied for their ability to inhibit plasmodium falciparum binding to red blood cells in culture. In the preliminary study, it was found that polymerizing the sialic acid gave an approximately 1000-fold enhancement in inhibition of the binding of parasite to cell, compared to monovalent sialyllactose. Similar to the foregoing example regarding the verotoxin blocking PLN, nanoparticles displaying the sialic acid arrays in conjunction with a second ligand, as described above, will be effective at blocking merozoites from attaching to infectable erythrocytes.

### Example 8

Candida Albicans Glycopeptide Presentation on PLN.

Uses of liposome carriers for vaccine development are of great interest. These types of vesicles can carry antigens and immunoadjuvants by either encapsulation or by surface-display.

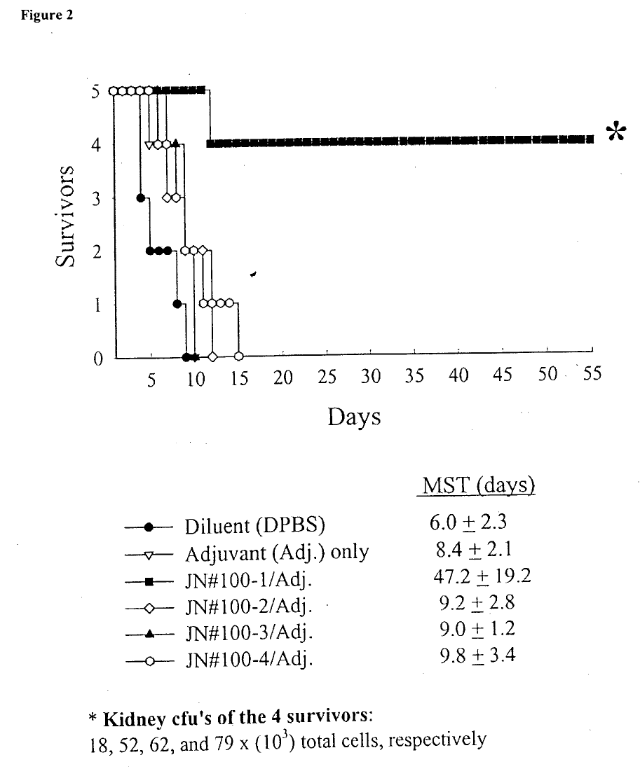

The phosphomannan peptide complex that comprises the cell wall components of C. albicans is a very weak immunogen but conjugation to BSA elicits increased antibody responses. Presentation of the phosphomannan peptide epitopes in a multivalent manner on the surface of PLN has shown a protective effect in mice when challenged with the pathogenic organism. A PLN formulated with monomers to which an anionic second ligand has been tethered, and consisting of about 10% phosphomannan peptidolipid complex administered via the peritoneal cavity, protected the test mice for fifty-five days, as seen in FIG. 2, after all of the un-PLN treated mice had died.

### Example 9

Candida Albicans Carbohydrate in Conjunction with T-Cell Directing Peptides as Presented on a PLN.

As reflected in the biomedical literature, a vaccine approach based on small peptides or carbohydrates has remained somewhat limited. This is likely related to their low immunogenicity and the scarcity of adjuvants that can be used with them in humans. Generally, small molecules act as haptens that lack the necessary Th epitopes to stimulate an effective immune response. Conjugation of small peptides or non-protein epitopes to other proteins, liposomes or polymer carriers has proven to be useful in stimulating antibody responses in a number of systems. The carrier serves a dual function, in addition to polyvalent peptide presentation, because it can also display a Th epitope. Long-lasting and potent immune responses have been elicited by small peptides covalently conjugated to the surface of the vesicle additionally carrying an adjuvant such as monophospholyl lipid A or lipopeptides such as Pam₃CAG. Liposome carriers that display separate B and Th epitopes can first target antigen-specific B-lymphocytes and, after uptake, the Th epitopes would then target intracellular MHC class 11-containing compartments. Such a synthetic construct induced a highly intense, anamnestic and long lasting (>2 years) immune response, in mice.

PLNs were prepared as described above that display small carbohydrate groups present in the phosphomannan peptide complex that comprises the antibody recognition region of the capsule of C. Albicans (see Example 8). These carbohydrate epitopes (β1-2 mannose di and trisaccharides or mannose-6-phosphate) as a first ligand may be presented in combination with B or Th epitopes. By this method, the elicitation of antibodies may be enhanced that will recognize key epitopes on the organism generating a specificity of response. However, formulating nanoparticles with a second ligand presenting an appropriately charged or hydrophobic (or hydrophilic) head group will produce a polyvalent binding unit that will substantially enhance the binding affinity relative to the nanoparticle displaying the phosphomannan peptide complex without such a second ligand.

### Example 10

Sigma Protein Displaying PLN for Targeting Antigen to the M-Cell.

Similar to the T-cell directing PLNs described in example 8, other nanoparticles may be formulated that display a another peptide (in this case, the Sigma peptide) on the surface of the PLN to direct the material to specifically target the M-cell. In this way surface antigens co-displayed with the sigma protein will be processed by the M-cell for a specific immune response. Likewise, material (such as DNA) entrapped inside an M-cell targeted PLN essentially will be invisible to the immune system until taken into the M-cell and processed. As in the previous example, formulating nanoparticles with a second ligand presenting an appropriately charged or hydrophobic (or hydrophilic) head group will produce a polyvalent binding unit that will substantially enhance the binding affinity relative to the nanoparticle displaying the Sigma peptide as a first ligand without such a second ligand.

### Example 11

Antibody Stimulation from EL-246 Peptide Presented on PLN.

Similar to the PLN that display epitopes found on Candida albicans for antibody generation, mice have been inoculated with PLNs that display EL-246 peptides. The expectation is that a new set of E and L-selectin neutralizing antibodies will be generated. Again, formulating nanoparticles with a second ligand presenting an appropriately charged or hydrophobic (or hydrophilic) head group will produce a polyvalent binding unit that will substantially enhance the binding affinity relative to the nanoparticle displaying the EL-246 peptide as a first ligand without such a second ligand. Technology for attaching a peptide antigen for C. albicans to a PLN carrier is described in copending U.S. patent application Ser. No. 09/076,833.

### Example 12

Library of Fucopeptide Analogs on PLN

A combinatorial library of glycopeptide analogs on solid support beads were developed to evaluate more economical and more selective mimetics of the sialyl Lewis X epitope toward selectin binding. “Panning” of the library on the bead support was proposed on the theory that selectin mediated adhesion is between one large surface (endothelial cell) and another large surface (leukocyte). In theory, the adhesion should work just as well between a selectin and a ligand-expressing bead surface.

However, it readily was found that the selectin bearing protein (selectin chimera) would not bind to a polymer bead that had any amount of the sialyl Lewis X covalently attached to the surface. Apparently, the polymer beads could not display the correct array of ligand structures in the right orientation or auxiliary charges required for selectin binding. Successful binding was achieved with beads that had sialyl Lewis X-bearing PLNs absorbed on them. Selectin chimera avidly bound to these “nanoparticle-coated” beads and because the chimeras were conjugated to a dye-precipitating enzyme, also colorized them in the process.

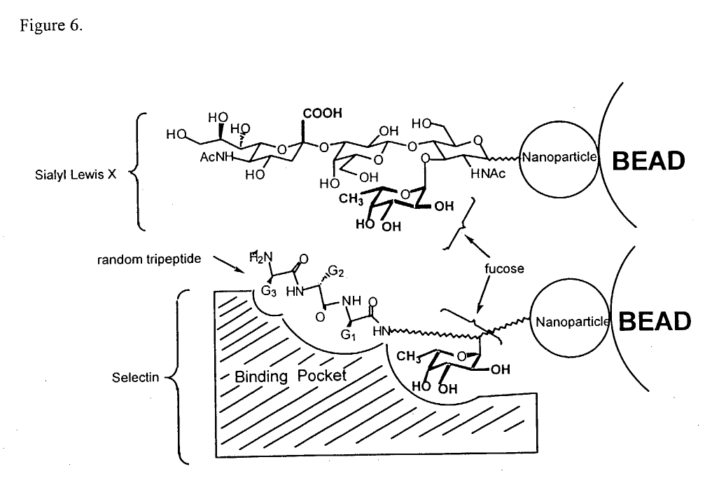

Based on these results, a library of structures was created based on bead bound PLN in order to determine whether the newly created, potential ligands would be displayed with the correct orientation for binding and have the “correct” anionic charges in the vicinity of the test “first ligand.” (See FIG. 6.) The mass of library beads was exposed to each of the three selectin chimeras sequentially and the enzyme then colorized any that tightly bound. The colored bead was then be removed by tweezers, washed and analyzed for the ligand structure.

The known key binding unit, the sialyl Lewis X structure—fucose—was held constant. It was intended that the rest of the sialyl Lewis X structure be represented with amino acids (a tripeptide) “growing” off of the fucose. Thus, a nanoparticle was created that presented the fucose unit with an adjacent amino group that would be extendable into random peptide sequences.

For such constructs, the 19 natural amino acids were used (minus cysteine) in both the D and the L form. Several readily available unnatural or rare amino acids, including sulfated tyrosine, were also incorporated into the library. All totaled, the beads displayed greater than 74,000 different tripeptide sequences. The first ligand (fucose-amine unit) percentage on the PLN was 5% of the total surface and sulfate coverage as charged head groups as a second ligand was 50%. The library was panned against the three selectins and 47 different tripeptide analogs were identified with some level of binding properties. For E-selectin, 3 sequences were identified; for P-selectin, 24 sequences were identified; and for L-selectin, 35 sequences were identified. In many of the sequences there was cross binding between two selectins and one showed cross binding between all three selectins. Quite a few sequences showed specificity for a single selectin. As with the nanoparticles in the foregoing examples, the polyvalent binding units substantially enhanced binding affinities.

### Example 13

Dendrimer Display of Selectin Blocking Compounds in Conjunction with Sulfate Groups.

Sialyl Lewis X (sLex)-like carbohydrates (3′-acetic acid, 3-fucosyllactose; 3′-sulfo, 3-fucosyllactose, 3′-sialyl, 3-fucosyllactose, or fucose) are covalently attached through a linker of about 4-30 atoms to a dendrimer polymer particle. Preferably, the polymers are spheres in the range of about 4 to 30 nm in diameter. The surface is substituted with 1 to 40% carbohydrate groups as a first ligand. The optimal substitution is 2-15%. The second ligand on the dendrimer surface are anionic charged groups at physiological pH (carboxylic acids, phosphates, sulfates, or hydroxamic acids). The substitution of monomers bearing anionic head groups for matrix monomers is in the range of 5 to 60%, with the optimal range being 15% to 35%. The additional anionic groups serve the function of supplying the charge of the sulfated tyrosines residues in the neighboring peptide backbone to the sialyl Lewis X glycosylation site on the physiological ligand (PSGL-1), shown to be crucial in P and L-selectin recognition. In preferred nanoparticles, the balance of the surface matrix may be made up of hydrophilic but chemically neutral groups such as hydroxyls.

The dendrimers are administered to an anesthetized mouse and the inhibition of rolling and sticking lymphocytes or leukocytes are measured in the vasculature of several tissues known to be models of selectin mediated cell recruitment (activated skin, activated mesentery, Peyer's patch). The activities of the materials are measured by a ProteoFlow apparatus to assess the percent reduction in rolling and sticking cell vs. the control and percent increase in the velocity of cells that do interact with the endothelium, vs. the control. The ProteoFlow Index (PFI) is used for evaluating the number of rolling/sticking cells as discussed above.

### Example 14

PLN with Entrapped PZP-1 Glycoprotein for Contraceptive Vaccine Delivery.

Porcine zona pellucida (PZP-1) is a glycoprotein found in the extracellular matrix surrounding oocytes and is important in fertilization and sperm recognition. It was found that monoclonal antibodies generated against this protein act as a short duration contraceptive in the treated animal. The duration of the protein in vivo make it necessary for the administrator to treat the animal multiple times per season to achieve year-long contraception.

PLNs having entrapped effective dosages of PZP-1 may be targeted to immune system cells with externally displayed polyvalent binding units as described above.

### Example 15

PS-76 Peptide Displayed Polyvalently on PNL.

Similar to the EL-246 peptide selectin blocking PLN described above, nanoparticles are constructed to display the peptide epitope identified through a phage library panning against antibodies that bind to carbohydrate epitopes found on C. albicans. The peptide epitope is used as synthetic mimetic to raise antibodies for vaccine generation utilizing nanoparticles bearing externally displayed polyvalent binding units as described above.

### Example 16

Inhibition of Shiga-Like Toxin (SLT) Binding to Daudi Cells by Carbohydrate Displaying Nanoparticles.

Shiga-like toxin (SLT) is the major causative agent of human toxicity by certain E. coli species notably O-157. The SLT is a pentameric protein excreted by the organism that binds to glycoproteins on sensitive cells causing hemorrhagic colitis and uremic syndrome. Daudi cells exposed to very low levels of toxin start to die after 12 hours. Nanoparticles displaying an analog of the carbohydrate recognized by the toxin (Gb₃) were able to completely block the toxin binding to cells. In addition to the carbohydrate groups, a second ligand to provide charge played a major role in the activity. Only head groups contributing an amino (basic) functionality led to an active formulation. A review of the key amino acids in the SLT binding sites indeed revealed the presence of essential acidic groups: aspartic acid 32, and multiple glutamic acid residues in positions 27, 30 and 130 (Maloney and Lingwood, J. Exp. Med. (1994) 180:191-201). This strongly suggests that in addition to arrayed carbohydrate recognition the amino groups are participating in binding to the acidic residues, to greatly augment to overall potency of the nanoparticle.

### Example 17

Shear Assay to Study in Vivo Inhibition of Selectin-Mediated Recruitment by Nanoparticles Displaying Carbohydrate Structures.

A). Peyer's Patch High Endothelial Venules.

A fully anesthetized mouse was incised to expose the abdominal wall to allow the small intestine to be externalized. The intestine was positioned for microscopic examination of the Peyer's patch. Coverslipping of the Peyer's patch allowed visualization of the desired region of the microvascular blood vessels and recording of the L-selectin mediated recruitment of lymphocytes. The lateral tail vein was then canulated to allow infusion of fluorescently labeled lymphocytes and experimental nanoparticle formulations. The adhesion of lymphocytes to the MadCAM-I ligand of the Peyer's patch is known to be strongly mediated by the (α4/β7 integrin in addition to L-selectin. In this experimental setting, to dissect the blocking of selectin-mediated adhesion from that of integrin mediated adhesion, the anti-(α4/β7 antibody PS-2 was injected alone, in controls, or in combination with the nanoparticle inhibitors to assess L-selectin dependent rolling. The percentage of rolling cells was determined as a fraction of all cells observed. The cell velocity was determined as delta time (seconds) observed for rolling cells in a 200-400 μm section of vessel.

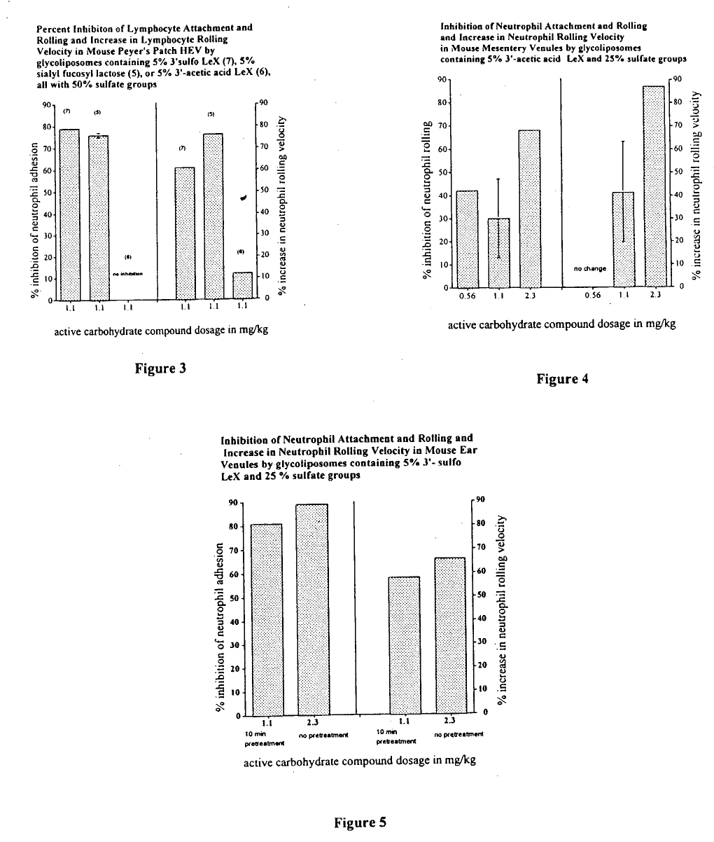

FIG. 3 shows a comparison of three different types of carbohydrates presented on a sulfated nanoparticle surface compared to the control treated with PS-2 antibody alone. FIG. 3 also shows the increase in cell velocity of each formulation relative to the control treated with PS-2 antibody alone. In these experiments, the lymphocytes were pretreated with the nanoparticle formulations for 10 min. prior to injection. The administration of these compounds corresponds to a 1.1 mg/kg dosage of carbohydrate.

B). Mesentery Venules

A variation on the Peyer's patch method was used to examine the P-selectin-mediated adhesion in a mouse mesentery venule model. A lateral incision was made into the abdominal wall to allow the small intestine to be externalized. The intestine was positioned for microscopic examination of the mesentery. A solution of PMA in phosphate buffered saline was superfused directly onto the mesentery to allow for upregulation of P-selectin in the endothelium of the mesentery venules. The area of the incision was treated and cannulation of the tail vein was performed as indicated above for Peyer's patch. A variable dose of carbohydrate- and sulfate-displaying nanoparticle was administered. The data analysis was the same as that described for the Peyer's patch model above.

FIG. 4 shows the reduction in number of leukocyte rolling and sticking events compared to the untreated control. FIG. 4 also shows the increase in cell velocity relative to the untreated control. At the highest dosage tested (2.3 mg/kg of carbohydrate), there was a greater than 65% reduction in the number of rolling/sticking leukocytes and a nearly 90% increase in the rolling velocity, over the control.

C). Cutaneous Venules

A variation on the mesentery venule method was used to examine the E and P-selectin mediated adhesion in a cutaneous recruitment model. A mouse ear pinna, both medial and lateral sides, was superfused with TNFα in DMSO prior to the experiment, to allow for upregulation of E-selectin in vascular endothelium. The mouse was then positioned on stage with medial side of the ear facing down on the silicone gel, to allow for visualization of microvascular blood vessels. Cells were then injected (as above), and adhesion and rolling events on the blood vessel endothelium were imaged. This experimental method was employed to examine E-selectin and PSGL-1 mediated adhesion events. A variable dose of carbohydrate- and sulfate-displaying nanoparticle was administered. The data analysis was the same as that described for the Peyer's patch model above.

FIG. 5 shows the reduction in number of leukocyte rolling and sticking events compared to the untreated control. FIG. 5 also shows the increase in cell velocity relative to the untreated control. Two ways of administering the nanoparticle formulations are compared. The neutrophils in the first instance were pretreated for 10 min. with a dose of 1.1 mg/kg (carbohydrate wt.) of nanoparticles. In the second the animal received a 2.3 mg/kg (carbohydrate wt.) dose without pretreated prior to injection. This demonstrates that neutrophil pretreatment gives an enhanced inhibitory effect on the neutrophil interaction with the E and P-selectin-expressing tissues. But, the benefits of pretreatment can be more than compensated for by increasing the nanoparticle dosage.

Additional References

1) J. O. Nagy and D. H. Charych, “Artificial Cell Membranes”, Chemtech, 1996, 26, 24.

2) W. Spevak, C. Foxall, D. H. Charych, F. Dasgupta, J. O. Nagy, “Carbohydrates in an Acidic Multivalent Assembly: Nanomolar P-Selectin Inhibitors”, J. Med. Chem., 1996,39, 1018.

3) Spevak, J. O. Nagy, D. H. Charych, “Molecular Assemblies of Functionalized Polydiacetylenes”, Advanced Materials, 1995, 7, 85.

4) W. Spevak, J. O. Nagy, D. H. Charych, M. E. Schaefer, J. H. Gilbert, M. D. Bednarski, “Polymerized Liposomes Containing C-Glycosides of Sialic Acid are Potent Inhibitors of Influenza Virus in vitro Infectivity”, J. Amer. Chem. Soc., 1993, 115, 1146.

5) J. Okada, S. Cohen, R. Langer, “In Vitro Evaluation of Polymerization Liposomes as an Oral Drug Delivery System”, Pharm. Res. 1995, 12, 576.

6) R. W. Storrs et al., “Paramagnetic Polymerized Liposomes as New Recirculating MR Contrast Agents”, J. Magnetic Resonance Imaging, 1995, 5, 719.

7) D. A. Sipkins, et al., “Detection of tumor angiogenesis in vivo by targeted magnetic resonance imaging”, Nature Med. 1998, 4, 623.

8) S. Takeoka, et al., “Phase Separation of Polymerized Mixed Liposomes: Analysis of Release Behavior of Entrapped Molecules with Skeletonization”, Macromol. 1991, 24, 1279.

9) B. M., Discher, et al., “Polymersomes: Tough Vesicles Made from Diblock Copolymers” Science, 1999, 284, 1143.

10) S. Takeoka, et al., “Control of Release of Encapsulated Molecules from Polymerized Mixed Liposomes Induced by Physical or Chemical Stimuli”, J. Controlled Release, 1989,9, 177.

11) S. Kobayashi, H. Uyama, “Polymeric Liposomes: Preparation, Characterization, and Applications”, Polish J. Chem., 1994, 68, 417.

12) H -H. Hub, et al. “Polymerizable Phospholipid Analogues-New Stable Biomembrane and Cell Models”, Angew. Chem. Int. Ed. Engl. 1980, 19, 938.

13) G. Lukowski, et al. “Acrylic acid copolymer nanoparticles for drug delivery. Part II: Characterization of nanoparticles surface modified by adsorption of ethoxylated surfactants” Colloid and Polymer Sci., 1993, 271, 100.

14) Y. Imanishi, et al., “Multivalent Ligands for Inducing Receptor-Receptor Interactions”J.M.S.-Pure Appl. Chem., 1994, A31, 1519.

15) S. Tetsui, et al., “Opiod receptor affinity of multivalent ligand system consisting of polymerized liposome”, Int. J. Peptide Prolein Res. 1996, 48, 95.

16) R. W. Storrs et al., “Paramagnetic Polymerized Liposomes: Synthesis Characterization and Applications for Magnetic Resonance Imaging”, J. Amer. Chem. Soc., 1995, 117, 7301.

17) M. Hasegawa, et al., “Mutual Recognition between Polymerized Liposomes. IV. Polysaccharide-Lectin System”, Biotech. Appl. Biochem., 1992, 15, 40.

18) H. Kitano, et al., “Mutual Recognition between Polymerized Liposomes: enzyme and enzyme inhibitor system”, Biochim. et Biophys. Acta., 1988, 942, 131.

19) H. Kitano, et al., “Mutual Recognition between Polymerized Liposomes. In. Association Processes between Avidin and Biotin on Polymerized Liposome Surfaces”, Biotech. Appl. Biochem., 1991, 14, 192.

20) C. L. King, et al., “Targeted Polymerized Liposome Contrast Agents” U.S. Pat. No. 5,512,294, Apr. 30, 1996.

21) J. O. Nagy et al., “Inhibition of Selectin Binding” U.S. Pat. No. 5,962,422 Oct. 5, 1999.

22) J. O. Nagy et al., “Inhibition of Selectin Binding” U.S. Pat. No. 5,985,852 Nov. 16, 1999.

23) Saiki, et al. “Functional role of Sialyl Lewis X and fibronectin-derived RGDS peptide analogue on tumor-cell arrest in lungs followed by extravasation” Int. J. Cancer: 65, 833 (1996).

24) Kitov, et al., Nature 403:669-672 (2000).

25) Armstrong et al. J. Infect. Dis. 164:1160-1167 (1991). 26) U.S. Pat. No. 6,004,534.

27) Tam, J. P. 1996. Recent advances in multiple antigen peptides. J. hnmunol. Methods 196:17-32.

28) Barr, I. G. and Mitchell, G. F. 1996. ISCOMs (Immunostimulating complexes): The first decade. Immunol. Cell Biol. 74:8-25.

29) Alving, C. R., Koulchin, V., Glenn, G. M. and Rao, M. 1995. Liposomes as carriers of peptide antigens: induction of antibodies and cytotoxic T lymphocytes to conjugated and unconjugated peptides. Immunol. Rev. 145:5-31.

30) Frisch, B. Muller, S. Briand, J. P. Van Regenmortel, M. H. and Schuber, F. 1991. Parameters affecting the immunogenicity of a liposome-associated synthetic hexapeptide antigen. Eur. J. Immunol. 21:185-193.

31) Friede, M., Muller, S., Briand, J. P. Van Regenmortel, M. H. and Schuber, F. 1993. Induction of immune response against a short synthetic peptide antigen coupled to small, neutral liposomes containing monophosphoryl lipid A. Mol. Immunol. 30:539-547.

32) Fernandes, I., Frisch, B., Muller, S. and Schuber, F. 1997. Synthetic Lipopeptides incorporated in liposomes: In vitro stimulation of the proliferation of murine splenocytes and In vivo induction of an immune response against a peptide antigen. Mol. Immunol. 34:569-576.

33) Boeckler, C., Dautel, D., Schelte, P., Frisch, B., Wachsmann, D., Klein, J -P. and Schuber, F. 1999. Design of highly immunogenic liposomal constructs combining structurally independent B cell and T helper cell peptide epitopes. Eur. J. Immunol. 29:2297-2308.

34) Aramaki, A. Y., Fujii, Y., Yachi, K., Kikuchi, H., and Tsuchiya, S. 1994. Activation of Systemic and Mucosal Immune Response Following Nasal Administration of Liposomes. Vaccine 12:1241-1245.

35) McCullough and H. N., Juliano, R. C. 1979. Organ-selective action of an anti-tumor drug: Pharmacological studies of liposome-encapsulated beta-cytosine arabinoside administered via the respiratory system of the rat. J. Natl. Cancer Inst. 63:727-731.

36) W. Rubas et al., “Incorporation of the reovirus M cell attachment protein into small unilamellar vesicles: incorporation efficiency and binding capability to L929 cells in vitro.” J. Microencapsulation (1990) 7, 385.

37) Tsai, et al. “Synthesis of sialyl lewis x mimetics using the Ugi four-component reaction” Bioorg. & Med. Chem. Lett. (1998) 8, 2333.

38) Tsukida, et al. “Studies on selectin blockers. 7. Structure-activity relationships of sialyl lewis x mimetics based on modified ser-glu dipeptides” J. Med. Chem. (1998) 41, 4279.

39) Sutherlin et al. “Generation of C-glycoside peptide ligands for cell surface carbohydrate receptors using a four component condensation on solid support” J. Org. Chem. (1996) 61, 8350 (describes a method for preparation of a combinatorial library of small selectin blocking glycopeptides).

40) Roy, et al. J. Chem Soc., Chem. Commun. (1993) 1869.

41) Roy, et al. Polymer News (1996) 21, 226.

42) Zanini, et al. Bioconjug. Chem. (1997) 8, 187.

43) Page, et al. Bioorg. Med. Chem. (1996) 4, 1949.

44) Zanini, et al. J. Amer. Chem. Soc. (1997) 119, 2088.

45) Ashton, et al. J. Org. Chem. (1998) 63, 3429.

46) Aoi, et al., Macromolecules (1995) 28, 5391.

47) R. G. Brown et al., “Evidence for a long-lasting single administration contraceptive vaccine in wild grey seals” J. Reproductive Immunol. (1997) 35, 43.

All documents, including texts, patents, patent applications and joumal articles identified herein are incorporated by reference in their entirety.

1. A nanoparticle comprising:
a carrier;
a first ligand displayed on said carrier; and
a second ligand, that is different than the first ligand, displayed on said carrier;
wherein said first ligand and said second ligand form a polyvalent binding unit that is effective to produce a specific interaction between the nanoparticle and one or more receptors on a target under physiologically relevant shear conditions;
and wherein said second ligand interacts specifically with said one or more receptors based on its charge or hydrophobicity.

2. The nanoparticle of claim 1, further comprising a third ligand displayed on the carrier that is different than said first ligand and said second ligand;
wherein said first ligand, said second ligand and said third ligand form a polyvalent binding unit that is effective to produce a specific interaction between the nanoparticle and one or more receptors on a target under physiologically relevant shear conditions.

3. The nanoparticle nanoparticle of claim 1, wherein said specific interaction produces a downstream event selected from the group consisting of endocytosis, cell signaling, phagocytosis, cell differentiation, apoptosis, cytolysis, transcriptional and translational events, and oxidative bursts.

4. The nanoparticle of claim 1, wherein said specific interaction modulates an event selected from the group consisting of cell—cell interaction, cell—extracellular matrix interaction, pathogen—cell interaction, and pathogen—extracellular matrix interaction, fertilization, inflammation, cancer metastasis, cellular migration, and pathogenic protein—cell interactions.

5. The nanoparticle of claim 1, wherein said receptor is selected from the group consisting of integrins, chemokines, cytokines and other inflammatory effector receptors expressed on mammalian cells and inflamed tissues.

6. The nanoparticle of claim 1, wherein the target is selected from the group consisting of leukocytes, epithelial cells, endothelial cells, epidermal cells, neurons, red blood cells, tumor cells, endocrine cells, dendritic cells, M cells, stem cells, osteoblasts, osteocytes, bone marrow cells and histiocytes (tissue macrophages).

7. The nanoparticle of claim 1, wherein said first ligand is selected from the group consisting of proteins; peptides (including D-, L- and cyclic peptides and nonnaturally occurring peptides); carbohydrates; antibodies and fragments thereof; toxins produced by pathogens; pathogen adhesion molecules; mammalian cell surface molecules; mammalian extracellular matrix molecules;
glycopeptides; glycolipids; peptidolipids; fucopeptides; and peptide, carbohydrate and small molecule mimetics of the foregoing; polynucleotides (including DNA and RNA); and derivatives of the foregoing.

8. The nanoparticle of claim 7, wherein said mammalian cell is selected from the group consisting of leukocytes, epithelial cells, endothelial cells, epidermal cells, neurons, red blood cells, tumor cells and endocrine cells.

9. The nanoparticle of claim 7, wherein said pathogen is selected from the group consisting of viruses, bacteria, fungi and parasites.

10. The nanoparticle of claim 9, wherein said fungi is selected from the genus Candida.

11. The nanoparticle of claim 1, wherein said specific interaction is selected from the group consisting of specific binding, target recognition, molecular address recognition, rolling adhesion, signal transduction and attachment adhesion.

12. The nanoparticle of claim 11, wherein said specific interaction is specific binding at an IC50 affinity of about 1 micromolar or less, and preferably one nanomolar or less, as measured under physiologically relevant shear conditions.

13. The nanoparticle of claim 11, wherein said interaction or downstream event is enhanced or is inhibited.

14. The nanoparticle of claim 11, wherein the inhibition of said rolling or attachment adhesion is characterized by a ProteoFlow Index (PFI) in the range of about 0.5 to 50.

15. The nanoparticle of any of claims 1 to 14, wherein said carrier is comprised of polymerized lipids.

16. The nanoparticle of claim 15, wherein said first ligand and said second ligand are spaced apart by lipids that do not carry a ligand or a charged head group.

17. The nanoparticle of claim 15, wherein said first ligand and said second ligand are covalently attached to the same lipid molecule by a single tether.

18. The nanoparticle of claim 15, wherein said first ligand and said second ligand are covalently attached to separate lipid molecules by a tether.

19. The nanoparticle of claim 18, wherein said tethers are about 5 to 50 atoms in length, preferably about 8 to 15 atoms in length, and most preferably about 10 to 12 atoms in length.

20. The nanoparticle of claim 15, wherein said nanoparticle has a diameter of less than about 200 nm, and preferably less than about 100 nm.

21. The nanoparticle of claim 15, wherein said second ligand is selected from the group consisting of head groups that are positively charged at physiological pH, head groups that are negatively charged at physiological pH, and head groups that have a neutral charge and are hydrophobic at physiological pH.

22. The nanoparticle of claim 15, wherein said first ligand is selected from the group of first ligands identified in Table 1 and said second ligand is selected from the group consisting of second ligands identified in Table 1.

23. The nanoparticle of claim 22 wherein said first ligand and said second ligand are selected from the first and second ligand pairs identified in Table 1.

24. The nanoparticle of any of claims 1-23 further comprising a biological attractor or targeting molecule selected from the group consisting of B-cell epitopes, T-cell epitopes, sigma -1 protein of a reovirus, invasin of Yersinia pseudotuberculosis, intimin of enteropathogenic Escherichia coli and Tir of enteropathogenic E. coli.

25. A therapeutic formulation in unit dose form comprising the nanoparticle of any of claims 1-24, in an amount that is effective to modulate a specific interaction between a cell or toxin and its receptor on a target in a host; wherein said first ligand is derived from or mimics a ligand on the cell or toxin.

26. The formulation of claim 25, formulated for oral, nasal, parenteral, buccal, mucosal or inhalation routes of administration.

27. A method for sequestration of toxins in the bloodstream of a host, comprising the step of administering an amount of the nanoparticle according to any of claims 1-24 that is effective to bind a circulating toxin or toxic metabolic product; wherein said first ligand is derived from or mimics a receptor that specifically binds to the toxin or toxic metabolic product.

28. The method of claim 27, wherein said binding is effective to produce an effect selected from the group consisting of decreased inflammation, decreased systemic toxicity, chelation therapy, neutralization of pathogens, inhibition of metastasis, inhibition of drug intoxication, tissue -regeneration, selective cellular differentiation and proliferation.

29. A vaccine comprising the nanoparticle of any of claims 1-24, wherein said first ligand comprises an epitope that is derived from a pathogen, pathogen-derived toxin or tumor cell and said vaccine is formulated to elicit a protective immune response against that pathogen or tumor cell.

30. The vaccine of claim 29, wherein the pathogen is selected from the group consisting of E. coli, Candida albicans, Brucella species, Salmonella species, Shigella species, Pseudomonas species, Bordetella species, Clostridium species, Group B strep, E. coli 0157, Brucella species, Norwalk Virus, anthrax, HIV, STDs, chlamidia, HBV, malaria, cell wall proteins of Candida species, and the GB3 toxin from E. coli 0157.

31. A diagnostic method comprising the steps of allowing a nanoparticle according to any of claims 1-24 to bind to a toxin or pathogen or an antibody against such toxins or pathogens and then detecting nanoparticles bound to such toxins, pathogens or antibodies.

32. A diagnostic imaging agent comprising the nanoparticle of any of claims 1-24 and further comprising a contrast agent detectable by medical resonance imaging or other visualization techniques.

33. A delivery vehicle comprising the nanoparticle of any of claims 1-24 and further comprising an agent to be delivered to the target.

34. The delivery vehicle of claim 33, wherein said agent is selected from the group consisting of therapeutic and cytotoxic agents; and wherein said agent is carried internally.

35. A method for optimizing a nanoparticle displaying polyvalent binding units having a first ligand and a second ligand, comprising the steps of:
optimizing the amount of said first ligand on the nanoparticle under physiologically relevant shear conditions; and
holding the optimized amount of said first ligand constant, optimizing the amount of said second ligand under physiologically relevant shear conditions.

36. A nanoparticle library comprising:
multiple polymer beads, wherein each bead contains a plurality of nanoparticles associated with the surface of said polymer bead;
wherein each nanoparticle comprises:
a first unique ligand displayed on said carrier; and
a second unique ligand, that is different from the first ligand, displayed on said carrier,
wherein said second ligand is selected from the group consisting of a positively charged head group, a negatively charged head group, and a hydrophobic head group, and
wherein said first and second ligands form a polyvalent binding unit; and,
wherein all of the nanoparticles associated with any one bead display the same polyvalent binding unit.

37. The nanoparticle library according to claim 36 wherein the multiple beads comprise nanoparticles comprising mutations to a known ligand.

38. The nanoparticle library according to claim 37 wherein said mutations are created using a combinatorial method of producing multiple variant molecules.

39. The nanoparticle library according to claim 38 wherein the mutations are specifically defined.

40. The nanoparticle library according to claim 36 wherein the multiple polymer beads are arranged in an array such that a physical distinction can be made between beads which comprise nanoparticles carrying distinct binding units.

41. The nanoparticle library of claim 36 wherein the nanoparticles further comprise detectable labels.

42. The nanoparticle library of claim 41 wherein a different detectable label is associated with those beads that comprise nanoparticles bearing distinct binding units.

43. The nanoparticle library according to any one of claims 36-42 wherein the first ligand comprises a known epitope.

44. The nanoparticle library according to any one of claims 36-42 wherein the first ligand comprises an immunoglobulin fragment.

45. A method of screening agents comprising adding at least one candidate agent to the nanoparticle library according to any one of claims 36-42 and detecting binding of the candidate agent to any one or more of the polymer beads in the library.

46. The method of claim 45 wherein the candidate agent comprises a detectable label.

47. The method of claim 46 wherein the candidate agent acts as a receptor to the first ligand.

48. A vaccine comprising the nanoparticle of any of claims 1-24, further comprising a polynucleotide sequence that encodes an epitope found on a pathogen or a tumor cell and the vaccine is formulated to elicit a protective immune response against that pathogen or tumor cell.

49. The vaccine of claim 48, wherein said first ligand comprises a targeting molecule that directs the epitope to antigen presenting cells contained in immune compartments of the mucosal system of an animal.

50. A vaccine according to any of claims 1-24, wherein said first ligand is selected from the group consisting of B-cell epitopes, T-cell epitopes, sigma-1 protein of a reovirus, invasin of Yersinia pseudotuberculosis, intimin of enteropathogenic Escherichia coli and Tir of enteropathogenic E. coli.

51. A method of deliverying nanoparticle therapeutic molecules to an animal which comprises administering a vaccine construct containing a nanoparticle displaying a first and second ligand that is effective to elicit a humoral and/or cell mediated immune resopnse.

52. The method of claim 51, further comprising an epitope or DNA encoding the epitope and the first ligand is a targetting molecule that directs the epitope or DNA to antigen presenting cells in the mucosa of an animal.

53. The method of claim 51, wherein the route of administration is by an oral, sublingual, buccal or rectal route.

54. A nanoparticle produced by the process of claim 35.
